(19) United States
(12) Patent Application Publication
(10) Pub. No.: US 2021/0017574 A1
(43) Pub. Date: Jan. 21, 2021

(54) METHOD FOR ASSESSING THE LETHALITY AND THE LEVEL OF CROSS CONTAMINATION CONTROL OF A PROCESS NON-INVASIVELY

(71) Applicant: Fremonta Corporation, Fremont, CA (US)

(72) Inventors: Eric Wilhelmsen, Milpitas, CA (US); Florence Wu, Milpitas, CA (US); Yongqing Huang, Newark, CA (US)

(21) Appl. No.: 16/933,720

(22) Filed: Jul. 20, 2020

Related U.S. Application Data
(60) Provisional application No. 62/876,429, filed on Jul. 19, 2019.

(51) Int. Cl. C12Q 1/686; A23B 4/22; A23L 3/3463; G01N 1/20

(52) U.S. Cl. CPC C12Q 1/686; A23B 4/22; G01N 2001/1062; G01N 1/2035; A23L 3/34635

(57) ABSTRACT

Methods and devices for non-invasively assessing lethality and/or cross contamination of a process. In some embodiments, an aggregating sampler is used, such as a fixture catcher, to obtain samples before, after and/or during the process. In some embodiments, an isolated packet of bacteria is exposed to the active elements of the process without contacting the product. In other embodiments, a method to measure lethality using microgenomic analysis is reported. In still other embodiments, a procedure is reported to use the knowledge from a microgenomic process to use direct qPCR for identified genera species to measure cross contamination. These metrics have special utility in the validation of wash water performance but may have utility is assessing process performance when unpackaged product is treated as for blanching and irradiation. Process performance can include verification of process delivery or for research.

### BACKGROUND

This Application is a Non-Provisional of and claims the benefit of priority of U.S. Provisional Application No. 62/876,429 filed Jul. 19, 2019, the entire contents of which are incorporated herein by reference.

### FIELD OF THE INVENTION

The present invention is directed to the determination of lethality and/or cross-contamination of a process and associated sampling approaches.

### DESCRIPTION OF THE RELATED ART

The measurement of lethality and cross contamination are measurements of the before and after load for the target organism(s) be they the pathogens, surrogates or synthetic surrogates. For lethality, one measures the before and after load on the same product to measure the number of organisms killed. Lethality is usually expressed as logs of kill assuming that the process is first order (e.g. a 5-log process as required for juice products). Cross contamination is the transfer of microbial load from a carrier (before) to catcher (after) such as from one leaf to another leaf in a salad wash line. Standardized units for reporting have not been established for cross contamination.

The measurement of lethality is well known in thermal processing. Various strategies have been developed for performing challenge studies and heat penetration studies that measure the lethality of these processes. These techniques have been extended to other high lethality processes such as exposure to UV, ultra-high pressure or cold plasma. These studies rely on enumeration of a bacterial load before and after processing. In these studies, this bacterial load is either an actual pathogen or a surrogate organism. This is not a problem given that in many cases, the organisms are in a closed container or there are other means of preventing the bacterial load from effecting the product stream and the processing facility with such an invasive process.

Research to develop thermal processes for a flowing system rely on modeling and heat penetration studies to develop processes to deliver the desired time and temperature rather than directly measuring the lethality of the process. In some instances, special process lines have been used for confirmation studies with an actual microbial load. In these cases, special care is required to ensure that the microbial load does not impact product production. The ability to make these extrapolations is built on the knowledge base of over two centuries of experience.

For a flowing low lethality system such as fresh cut produce washing systems and other similar processes, measuring lethality is hindered by the lack of fundamental knowledge regarding the control parameters and the difficulties in recreating the process conditions in an environment suitable for an invasive microbial load. Some have proposed using synthetic surrogates, but one can never be sure these will perform as the target microorganism in the special environment of the wash system.

Efforts to use the wild flora with Aerobic Plate Count (APC) or total coliform count have been frustrated by the variability of the measures on the before and after product. Very large data sets show promise for demonstrating the trends but are not suitable as a metric for process evaluation. Furthermore, the traditional plating techniques require long incubation time. Thus, there is a need for improved methods for assessing lethality and/or cross-contamination.

### BRIEF SUMMARY

In one aspect, the invention pertains to methods that provide improved assessment of lethality and/or cross-contamination of a process, preferably non-invasively and close to real-time. Specifically, the methods can include non-invasively measuring the lethality of wash lines and related processes that can by extension be used to measure cross contamination control, preferably in close to real-time.

In one embodiment of the present invention, an isolated packet of bacteria is exposed to the active elements of the process without contacting the product. In another embodiment, a method to measure lethality using microgenomic analysis is reported. In third embodiment, a procedure is reported to use the knowledge from a microgenomic process to use direct qPCR for identified genera species to measure cross contamination. These metrics have special utility in the validation of wash water performance but may have utility in assessing process performance when unpackaged product is treated, such as for blanching and irradiation. Process performance includes verification of process delivery or for research.

In one aspect, the invention pertains to the use of a limited permeability packet enclosing microorganisms to measure lethality.

In another aspect, a reference enumeration is used to convert percentage determinations from metagenomic analysis to actual enumerations.

In still another aspect, results from the above noted enumerations are used to identify genera or species to use for direct qPCR.

In yet another aspect, swabs (e.g. MicroTally™ swab) are used to reduce uncertainty as to lethality and/or levels of contamination. The swabs can include any absorbent or adsorbent material. In some embodiments, the swab can be configured as a sheet or cloth with a suitably sized sampling surface and can be manually applied or can be held within a fixed stationary sampling device to act as a fixed catcher.

It is appreciated that the concepts of the invention described herein can be incorporated, partly or fully, with any of the approaches described in any of the following disclosures, incorporated herein by reference in their entireties for all purposes: PCT Application No. PCT/US2018/045699 filed Aug. 8, 2018, entitled “Method and Apparatus for Applying Aggregating Sampling to Foods;” U.S. Non-Provisional application Ser. No. 16/525,350, filed Jul. 29, 2019, entitled “Method and Apparatus for Applying Aggregating Sampling to Foods;” and U.S. Non-Provisional application Ser. No. 16/859,528 filed Apr. 27, 2020 entitled “Powered Sampling Device and Methods.”

### BRIEF DESCRIPTION OF THE FIGURES

Embodiments of the present invention can be further understood by referring to the following figures depicting methods in accordance with the present invention. These figures illustrate certain aspects but should not be considered limiting of the scope of the invention.

FIG. 1 shows a flow chart illustrating a method of measuring a lethality of a process that is compatible with use during commercial food processing operations, in accordance with some embodiments.

FIG. 2 shows a flow chart illustrating another method of measuring a lethality of a process that is compatible with use during commercial food processing operations, in accordance with some embodiments.

FIG. 3 shows a flow chart illustrating a method of measuring cross contamination that is compatible with use during commercial food processing operations, in accordance with some embodiments.

### DETAILED DESCRIPTION

The process of measuring lethality and cross contamination are measurements of before and after microbial loads for target organisms, usually pathogens but surrogates can also be considered targets. This is normally done with some type of inoculation. The methods and procedures taught herein can avoid inoculation and permit these studies to be done during commercial production allowing the measurement of process performance under actual processing conditions. It is further taught how to use the accumulated knowledge of various wash system and process combinations to reduce the cost and increase the speed to result. Each product and wash system can be expected to perform somewhat differently so it is not a one size fits all solution.

These assays can be used to validate processes as they are metrics of performance. These assays can be used to verify that processes are performing as a verification tool. These assays can be used to direct process improvement research or to compare alternative processes. As with most assays, as they become more widely accepted, there will be more uses. These categories of use are only examples. The present invention addresses three deficiencies in the traditional approach, speed, avoiding the introduction of bacteria into the product stream, and uncertainty in the measurements. Additionally, the discussion will address calibration of correlated data. To aid the reader, each of these areas will be addressed individually and then some specific embodiments will be elaborated.

The speed to result can tremendously impact the value of information. Often delays in receiving information can delay critical responses. Traditional plating techniques can take hours and in some cases days to develop. This pressure is partly responsible for the migration to molecular methods for many microbiological tests. Molecular methods are also easier to automate, thereby reducing labor. Therefore, although traditional plating and culture techniques can be used to practice the invention, the bulk of the discussion will focus on molecular methods with the goal to deliver results in less than 6 hours and more preferred would be less than 2 hours.

The time to result for most traditional approaches is delayed by an enrichment step. An enrichment step provides both dilution of inhibiting materials and an increase in concentration of the target(s). The shift from 5-10 cells of the target organisms to as much as 10⁶ cells in some cases renders the detection step much easier. However, the enrichment step prevents enumeration unless a Most Probable Number (MPN) procedure is used which would greatly increase costs and only increases the time to result.

Speed can be achieved in special cases using spectral measurement techniques where there is already sufficient signal strength because the target organisms are abundant. These optical approaches are beyond the scope of the present discussion and are in a phase of rapid improvement. Using one of these approaches, APC can be measured in situ given the high concentration of bacteria. The speed and low cost of such an analysis can offset the variability in APC with a large number of analyses. In a second category of this special case, large numbers of a bacteria exposed to process system but not exposed to the product stream may also be analyzed by spectral means. Such samples may be especially suited to this type of analysis given that both the concentration of organisms and the isolation from potentially interfering materials will enhance the spectral signal. In both of these categories, researchers are developing dyes and stains that enhance the sensitivity of the spectral methods.

As a final comment about speed to result, use of abundant wild flora avoids the time necessary for growth, unless as discussed below, one isolates the target organism from the product while still allowing contact with the treatment. Furthermore, it is necessary to use organisms that are abundant enough to measure changes in the before and after results.

Continuing onto the next topic, avoiding the introduction of bacteria into the product stream. The first approach is the use of the wild type organisms, the bacteria that are already present. This is challenging because as discussed, the total population is highly variable and includes organism that have a wide range of sensitivities to the various processes. Spore forming bacteria are highly resistant to almost all processes. Other genera, such as Pseudomonas, are more resistant to chemical treatments. These types of resistance obscure the signal for lethality and cross contamination, thus making cross contamination and lethality more difficult to quantify.

If one selects to use a portion of the wild type organisms in spite of the above challenges, one should first know what subset of organisms to test to be able to enumerate this subset. In a new system, where scant knowledge is available about the expected population, one may use metagenomic analysis to determine the relative abundance on the various genera of bacteria present using next generation sequencing techniques. The sequencing of the 16S ribosomal RNA is the current method of choice, although it is appreciated that any suitable method could be used. Additional sequence data can increase the resolution of the characterization, but this is not typically needed as genera are relatively homogenous with regards to process sensitivity. This metagenomic analysis only gives relative abundance of the various genera that are present which is suitable for identifying candidates for monitoring but is not suitable for measuring lethality or cross contamination. Those genera which greatly decline in percent abundance when comparing the population of raw and processed samples are sensitive to the process and are therefore candidates to monitor process performance. To convert this abundance information to relative abundance, one may enumerate a genus of bacteria in the before and after samples such a Pseudomonas or Bacillus to use as a normalizing factor to obtain relative numbers. This procedure can be done for any process and product. It is robust and powerful, but it is slow and costly. The enumeration if done by traditional plating will be the rate limiting step. However, increasingly qPCR kits are becoming available so the traditional enumeration can be replaced with a faster molecular procedure. The costs of metagenomic analysis continue to fall but presently, the two population profiles and enumerations can prove prohibitive for routine measuring of these process metrics but may be suitable for research purposes. The costs for this process declines sharply with the number of samples so that scale can make this a viable process even at current prices. This discussion will turn to the problem of calibrating the sensitivity of a surrogate to that of the more desirable pathogen targets below as a fourth topic of discussion.

After a system has been studied with the above system, one may use qPCR to directly enumerate the before and after abundance of the target genera or in some cases species of bacteria. It is not necessary to restrict the analysis to one genus if there is a small pool of genera that have appropriate responses to the process. This choice will be driven by the specific process under study or being monitored. Clearly, the costs of two qPCR will be less than the cost of two qPCR and two metagenomic analyses unless the marketplace has artificially skewed the costs.

The use of wild type bacteria by either of these approaches is expected to remove uncertainty from the determinations of lethality and cross contamination relative to using total bacterial populations but it may not be enough in some cases. Two alternative approaches are taught that will overcome the uncertainty but introduce an additional level of complexity to the calibration process presented below. Also discussed below are some tools to address the uncertainty directly.

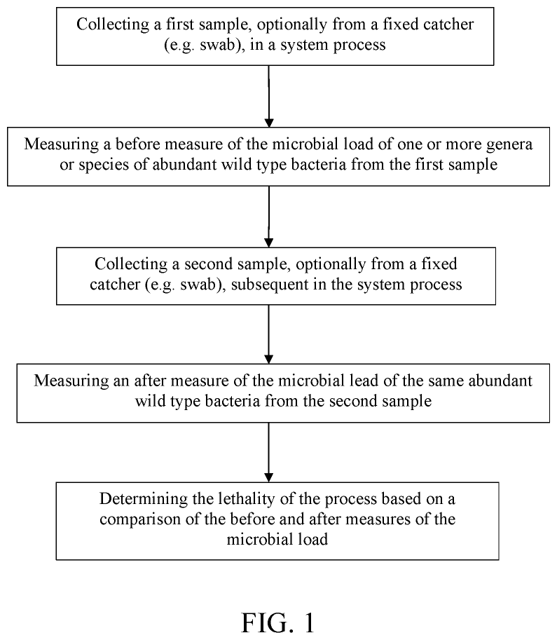

FIG. 1 illustrates such an example method of measuring lethality of a process by using wild type bacteria. As shown, the method includes steps of: collecting a first sample, in a system process; measuring a before measure of the microbial load of one or more genera or species of abundant wild type bacteria from the first sample; collecting a second sample, optionally from a fixed catcher (e.g. swab), subsequent in the system process; measuring an after measure of the microbial lead of the same abundant wild type bacteria from the second sample; and determining the lethality of the process by comparing the before and after measures of the bacteria. In some embodiments, determining the lethality includes reporting the log of the ratios of abundance as the lethality of the process in regard to the target organisms. The first sample can include one or more samples, and the second sample can include one or more samples. One or both of the first sample can be obtained from a fixed catcher. In some embodiments, the capture materials can be a fixed catcher, such as a swab or any suitable material. The fixed catcher can include any materials, device or components described in the examples in U.S. Non-Provisional Application No. 16/525,350, filed Jul. 29, 2019, incorporated herein by reference, although it is appreciated that various other configurations can be realized as well.

As mentioned above, as an alternative to using wild type bacteria, two approaches are taught to exposing target bacteria to the process conditions that do not expose the product stream to contamination from these surrogates except in the case of catastrophic failure. Conceptually, a suitable population of the target bacteria can be packaged to retain the bacteria and permit the conditions of the process to contact the target bacteria. This can be accomplished by packaging the bacteria in various semi-permeable membranes. The only requirement is retention of the bacteria and allowing the process agents to contact the bacteria. For a gaseous process, the permeation can be a solubility property and therefore have no true pores. For a liquid process such as an oxidizing sanitizer including, but not limited to, chlorine, ozone, peroxide, and other active oxygen species, the permeability can be afforded by small pores, generally less than 1 micron which will not permit the bacteria to pass. The use of a 0.22-micron pore size is preferred so as to provide a larger margin of retention. This pore size is used to filter sterilize. Typically, the pore size should be large enough to allow the process agent to passively contact the target bacteria in a reasonable time. As discussed in the embodiments below, it may be appropriate to provide a very permeable overwrap to prevent physical damage.

In some systems, active transport may be required because diffusion through the membrane is not fast enough for measuring the process performance. In such cases, the diffusion process can be accelerated with pressure. For liquid systems it may be necessary to use a swept surface configuration to prevent fouling. Even a few pounds of pressure will greatly accelerate diffusion if there is no back pressure on the other side of the packet. For this configuration to work, both sides of the packet need to be semi-permeable. When diffusion is acting passively, there may be cases where one side of the packet can be another material. In such cases, this other side may provide more resistance to mechanical damage.

The selection of organisms for use in these semipermeable packets with or without active transport is flexible. Given that they will be used in an operating food plant, the use of a nonpathogenic surrogate such as a Lactobacillus would be a fair choice. If there were a catastrophic failure the released organism would be an acceptable incidental contaminant. There are a number of candidate organisms that have been suggested for the various food pathogens. Generic E. coli would be another reasonable candidate. It is well studied and easy to measure by many different means. In addition, specific strains that present a worst case scenario such as resistance to sanitizer could be selected to strengthen the validity of study. Given that the organisms are segregated from the product even the actual pathogens could be considered. However, attenuated strains are a better choice for safety reasons. The consequences of an unplanned release can be deadly. Ultimately, the choice will be made in light of what measurement is to be made and the quality of the calibration which is discussed below.

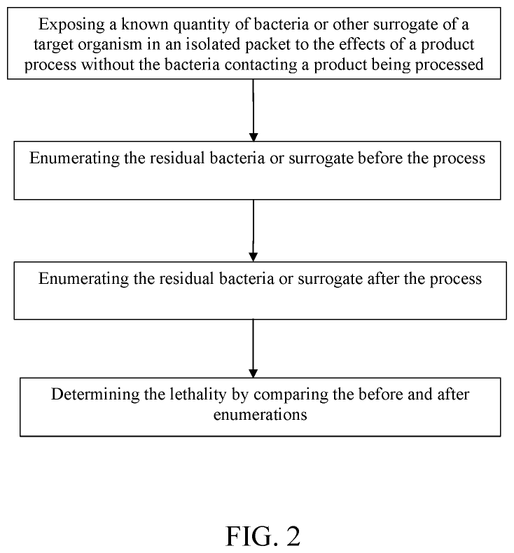

FIG. 2 illustrates an example of such a method of measuring lethality of a process by exposing a target bacteria to the process without the bacteria contacting the product. As shown, the method includes steps of: exposing a known quantity of bacteria or other surrogate of a target organism in an isolated packet to the effects of a product process without the bacteria contacting a product being processed; enumerating the residual bacteria or surrogate before the process; enumerating the residual bacteria or surrogate after the process; and determining the lethality by comparing the before and after enumerations. In some embodiments, determining the lethality includes reporting the lethality as the log of the ratios of the before and after enumerations. As described previously, a packet with a semi-permeable membrane can be used to isolate the target bacteria from products with the process, however, any suitable isolation means can be used.

There are four approaches to addressing the uncertainty in determinations of either lethality or cross contamination. The selected approach will affect the cost and speed to result. The first approach is the traditional approach of repeated measures. If you repeat a measure multiple times, the uncertainty becomes more manageable. Unfortunately, the uncertainty decreases as the square root of the number of tests which often makes this approach somewhat prohibitive in terms of cost. However, this approach is feasible and can even be desirable under some conditions if the measurements are important enough. This approach becomes considerably more acceptable when a spectral assay can be employed which can be close to instantaneous and has a very low cost.

The second approach for addressing uncertainty is closely related. One can increase the sample size. If a larger amount of product is made homogenous, an average value can be obtained that is more representative to the total population. In practice, this approach is limited by the ability to handle the materials. In the typical laboratory a few hundred gram is a very large sample. Normal practice for an extraction procedure is 25 to 50 grams. A five to ten-fold increase in sample size can be very helpful. Most commonly, a practitioner will focus on the extraction step where the bacteria are eluted into a suspension which can be made very homogenous as long as the organism to be enumerated is not so rare as to be less than 5 CFU in the sampled volume. Less than this level, the Poisson distribution should be considered as it becomes increasing likely that no organisms will be found in the aliquot. One should consider the potential to render to product to be extracted more homogenous as well. This is especially useful where there is a step that blends together and mixes product before washing. Various cutting and chopping operations are examples of these kinds of processes.

A third approach for dealing with uncertainty is to use an aggregating sampler, such as a swab (e.g., MicroTally™ Swab) which surface samples a large quantity of product albeit at reduced efficiency but providing a more representative sample of the microbial population. These can be used to collect before and after samples for either cross contamination or lethality studies. The ability to serve directly as a catcher for cross contamination is especially useful.

In some embodiments, the methods can utilize a fixed catcher. It is noteworthy that using a fixed catcher simplifies the measurement of cross contamination relative to the normal practice of running the catcher through the entire process. The fixed catcher need only be suspended in the product where it is in intimate contact with the process, generally the wash water, and subject to incidental contact with the product. In many cases, these two transfer mechanisms are the most important drivers of cross contamination. The fixed catcher avoid the problems of recovery and separation that make cross contamination measurements difficult for research and very difficult for in plant studies.

In some cases, alternative catchers have shown the potential to increase the sensitivity to measure cross contamination. These materials have been more similar to the product than swabs with surfaces thought to have protective niches, recesses, or openings that protect transferred bacteria from the sanitizer. Examples include rice paper, dried lotus leaves, and dried spinach leaves. These materials also have reducing potential that can neutralize oxidizing sanitizers which may further enhance the cross contamination signal.

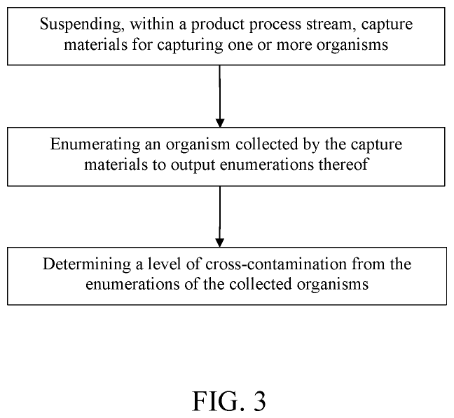

FIG. 3 illustrates such a method of measuring cross-contamination by suspending capture materials within a product process stream. As shown, the method can includes steps of: suspending, within a product process stream, capture materials for capturing one or more organisms; enumerating an organism collected by the capture materials to output enumerations thereof; and determining a level of cross-contamination from the enumerations. In some embodiments, the determination of the level of cross-contamination is performed by charting the enumerations as a measure of relative cross-contamination. In some embodiments, the capture materials can be a fixed catcher, such as a swab or any suitable material. The fixed catcher can include any materials, device or components described in the examples in U.S. Non-Provisional application Ser. No. 16/525,350, filed Jul. 29, 2019, incorporated herein by reference, although it is appreciated that various other configurations can be realized as well.

A fourth strategy for dealing with the uncertainty with using wild type organisms for determinations is to use the results of the metagenomic analysis to select specific genera or species. These organisms can be analyzed by qPCR even if they cannot be cultured or enumerated by traditional plating techniques. Such choices become apparent as systems become better characterized.

Turning to the topic of calibration, one should first consider the necessary degree of calibration for the intended purpose of the assay. Little or no calibration is needed if the goal is verifying the normal operation of a process. In fact, one can in many cases ignore the before measurement and only measure the after measure for control charting to assure that the entire process remains in control. The value of these metrics can be improved by showing that changes in response correlate with process effectiveness, but this additional data is not required for some process verification activities.

Working directly with pathogens in a pilot plant setting is generally not practical and presents hazards that should be avoided. Therefore, model systems comparing pathogens to appropriate surrogates such as closely related species, or similar species from other genera or even abiotic surrogate is the first step. There is substantial literature in this area, and it is beyond the scope of this document. Suffice it to say, that many benign bacteria are reasonable surrogates depending on purpose.

For process validation activities and some research activities, it is important to know that the observed results have some relationship to process effectiveness on the actual pathogens. In these later cases, there are two steps in the calibration to be considered. The need for both steps needs to be considered for all classes of process and membership in a class should not be assumed. In these cases, it is important to identify an organism or genera of organism that has similar sensitivity to a process as the pathogen of interest. Furthermore, one should establish a quantitative relationship between the sensitivity of the surrogate and that of the pathogen. Concepts such as reducing the surrogate to non-detect levels inherently yield quantitative data reflecting the sensitivity of the assay which in large measure is a function of effort and cost.

If it has been shown that a pathogen is sensitive to a particular process in a model system, it can be useful to use the metagenomic strategy discussed above to identify a sensitive wild type organism or sensitive genus. This genus can be studied in the model system to refine the relationship between changes in the surrogate population

To better illustrate the concepts described above, exemplary embodiments are provided as follows:

### Example 1

### Validating Water Treatment in Canal

Irrigation water in small canals is highly variable over time. Various treatments strategies are in use, but traditional validation is tedious requiring many 100 ml samples which are typically analyzed for coliforms or E. coli. Replacing the water samples with aggregating samplers such as a swab (e.g., MicroTally™ Swab) exposed to the water for 10 to 20 minutes will provide a more representative sample. These swabs can be placed in the water flow of the canal before and after the treatment location and be used to calculate lethality of the process. Each swab can be analyzed by traditional methods or can be analyzed molecularly or by spectral means given the relatively uniform background of the swabs. Sensitivity can be increased by concentrating the extracted organisms by centrifugation, filtration, absorption or other binding methods.

### Example 2

### Verification of Cross Contamination Control

Verification of cross contamination control can be used to confirm that the process control strategies are yielding the expected process for a fresh cut processing line. Given that only deviations for the norm need to be detected, it may not be necessary to collect the before data from the feed material and the resulting data can be control charted with an X-bar chart to detect deviations in the usual manner. For this procedure, wild bacteria are used as introducing surrogates into a commercial operation is undesirable. The swabs can be suspended in the wash stream to contact product and water borne bacteria for between 2 and 10 minutes to assess the cross-contamination pressure. The residual sanitizer of the swabs needs to be immediately neutralized; 50 mg of sodium thiosulfate in solutions has proven effective for this purpose. The APC, total coliforms or E. coli levels from the swabs can be control charted, but these metrics often lack sensitivity due to the variable abundance of organisms that are less effected by the wash sanitizers or by the low natural abundance of the target organisms. However, it has proven useful in some instances. Monitoring Lactobacillus levels, as learned through the metagenomic analysis approach outlined above, on the raw material and from the aggregating sampler ratio can be more sensitive to changes in cross contamination control. The verification is completed by control charting the ratio of these two numbers.

### Example 3

### Verification of the Lethality of a Wash Process

To verify the lethality of a wash process, a pre-determined population (e.g., 10 million) of viable cells of suitable organism is applied to a small carrier, for example a disc of non-woven poly olefin cloth, a food grade material, that is then sandwiched between two 0.22 micron polypropylene membrane filters that are sealed around it. The verification process can use many of these packets. Each packet is placed in a mesh bag to provide mechanical protection and means of restraining the packet.

The packets are suspended in the wash stream for a consistent amount of time, between 10 and 60 minutes, depending on the resolution that is desired. A positive control is suspended in distilled water as a recovery reference. The ratio of treated to positive control is control charting to allow verification of lethality for the process.

The enumeration of the organisms given that are in pure culture on the cloth in large numbers can be done in a variety of ways including direct spectral analysis, qPCR with appropriate primers, and traditional plating. The plating media can be a simple non-selective media given that a pure culture was used. The method of enumeration can be selected based on the need for speed to result.

### Example 4

### Validation of Cross Contamination Control

Using metagenomic analysis of the raw product and processed product with a reference enumeration, such as total Pseudomonas, identifies the species or genus (or genera) that will be monitored as a surrogate for the pathogens that might be present that are a cross contamination risk. It is helpful to obtain primers such that qPCR can be used rather than traditional plating methods which can be difficult if the surrogates are not readily cultured.

Using an appropriate aggregating sampler such as a swab (e.g., MicroTally™ Swab), suspended in the wash stream during an appropriate portion of the validation window, usually between 5 and 20 minutes of the 4-hour window, collects the organisms transferred from the product. Collect representative raw sample as reference. Confirm that the ratio of transferred organisms per swab to the concentration on the product meets the targeted specification. Preferably, this specification includes the time of sampler exposure. In some embodiments, it also includes the quenching procedure such as the one given in Example 2.

### Example 5

### Validation of the Lethality of a Wash Process

Assuming a lethality specification for the process, a log reduction level, one can confirm that this is met over a validation window at some level of confidence. The concept of complete kill is unachievable given that kill is a first over order process that asymptotes towards zero. The regulatory guidance has not provided a guideline at this time except the general requirement to do as good as possible. The appropriate window can be determined by assessing the window that includes a larger percentage of the observed variance. For a typical wash process this is about 4 hours. One should make enough measurements to achieve the desired confidence over this window. Practically speaking, in many cases, this is probably more than 12 but less than 25 individual determinations.

For the system in question, bench scale work using metagenomic techniques and pathogenic inoculation is used to establish the relative sensitivity of the target pathogen and a wild type surrogate. This data will be used to generate a correlation curve between the pathogen lethality and the observed lethality of the surrogate under the conditions of the process. This is a multi-step process where the sanitizer concentration and times are varied in test mixtures. The pathogens need to be applied to the product surface as this affords some protection that needs to be included. The goal is to have a ratio of a kinetic factor such as the first order rate constant or half kill under the process conditions.

Assuming the wild type surrogate is abundant enough, one can use aggregate sampling and direct qPCR to measure the before and after levels of the surrogate and then calculate the lethality. One should consider if all measures need to meet the goal, if the average must meet the goal or if two goals are appropriate. These decisions are beyond the scope of this example.

Wild bacteria is selected that reacts to the process predictably and reliably similar or proportional to the target pathogen. Examples of wild bacteria that can be utilized in the above-described methods include, but are not limited to: Acidovorax, Acinetobacter, Aeromonas, Arthrobacter, Bacillus, Bacteroides, Calothrix, Chryseobacterium, Citrobacter, Clostridium, Comamonas, Cupriavidus, Enterobacter, Erwinia, Exiguobacterium, Flavobacterium, Janthinobacterium, Klebsiella, Massilia, Microvirus, Paenibacillus, Paracoccus, Pseudarthrobacter, Pseudoduganella, Pseudomonas, Psychrobacter, Rheinheimera, Rhizobium, Rhodococcus, Serratia, Sphingobacterium, Stenotrophomonas, Thermogemmatispora.

Surrogates are nonpathogenic alternatives for the pathogen of concern that react predictably and reliably similar or proportional to the target pathogen. Typically, the surrogates have similar or stronger survival capabilities under the conditions being validated. Surrogates can be biological or chemical. Examples of biological surrogates include, but are not limited to: Escherichia coli and its physiologically or genetically modified strains; Non-pathogenic and physiological or genetically modified Salmonella; Listeria species; and Lactic acid bacteria. The Lactic acid bacteria can include, but is not limited to, species in the genera of: Aerococcus, Enterococcus, Lactobacillus, Pediococcus, Lactococcus, Lactovum, Okadaella, Streptococcus, Leuconostoc, Weissella. Chemical surrogates can include any chemical agent that when exposed to an antimicrobial agent (e.g. chlorine) will react predictably proportional to the behavior of the target pathogens.

Pathogens for which lethality and contamination is being determined in the methods described above can include but is not limited to: Pathogenic E. coli (including EHEC and STEC), Salmonella, and Listeria monocytogenes.

It is appreciated that the concepts described herein are not limited to the above-noted examples and can be incorporated, in part or fully, within various other approaches and sampling methods. Further, it is appreciated that these concepts are pertinent to any field in which it is desired to provide an assessment of lethality or cross-contamination of a process. For example, the methods can be used in any food-related process as well as various other non-food related or industrial processes where the presence of pathogens or contamination is of concern.

In the foregoing specification, the invention is described with reference to specific embodiments thereof, but those skilled in the art will recognize that the invention is not limited thereto. Various features, embodiments and aspects of the above-described invention can be used individually or jointly. Further, the invention can be utilized in any number of environments and applications beyond those described herein without departing from the broader spirit and scope of the specification. The specification and drawings are, accordingly, to be regarded as illustrative rather than restrictive. It will be recognized that the terms “comprising,” “including,” and “having,” as used herein, are specifically intended to be read as open-ended terms of art.

1. A method for measuring a lethality of a process that is compatible with use during commercial food processing operations, the method comprising:
obtaining a before measure of microbial load of one or more genera or species of abundant wild type bacteria selected to serve as surrogates for one or more target organisms;
obtaining an after measure of microbial load of the same abundant wild type bacteria; and
reporting the log of the ratios of abundance as the lethality.

2. The method of measuring lethality of claim 1 where an aggregating sampler is used to collect sample of the abundant wild type bacteria for enumeration from which the after and/or the before measures are obtained.

3. The method of measuring lethality of claim 2 where the aggregating sampler is one or more fixed catchers.

4. The method of claim 1 where relative metagenomic levels and a reference enumeration are used to measure either or both of the before and after measures of microbial load.

5. The method of claim 1 where thiosulfate is used to quench residual sanitizer for analysis.

6. The method of claim 1 where metagenomic studies are used to identify targets which are then enumerated by direct qPCR.

7. A method for measuring a lethality of a process that is compatible with use during commercial food processing operations comprising:
exposing a known quantity of bacteria or other surrogate to the effects of the process without contacting the product;
enumerating the residual bacteria or surrogate; and
reporting the lethality as the log of the ratios of before and after enumerations.

8. The method of claim 7 further comprises contacting a separation or barrier, wherein the separation or barrier to contact is a semi-permeable membrane.

9. The method of claim 8 where the barrier is a filter with pores smaller than 2 microns, smaller than 1 micron, smaller than 0.75 microns, or smaller than 0.45 microns.

10. The method of claim 7 where the barrier is a bag or envelope.

11. The method of claim 7 where the semi-permeable membrane is configured to allow exposure of the bacteria or other surrogate to the process while preventing exposure of an external environment of the bag or envelope to the bacteria within.

12. The method of claim 7 where the enumeration is done by qPCR or direct spectroscopy of either an extract or in situ on a support of known reflectance.

13. A method for measuring cross contamination that is compatible with use during commercial food processing operations comprising:
suspending one or more organism capture materials in the process stream to obtain a sample;
enumerating an organism collected by the one or more capture materials to output enumerations; and
charting these enumerations as a measure of the relative cross contamination.

14. The method of claim 13 where a before value of the microbial load is measured as an index of cross contamination pressure and the log of the ratio of before to the in-process sample is reported as the level of cross contamination on control.

15. The method of claim 13 where the organism capture material is an aggregating sampler.

16. The method of claim 15 where the aggregating sampler includes a fixed catcher.

17. The method of claim 16 where the fixed catcher is a material with niches, recesses, or openings that act as cross-contamination catchers.

18. The method of claim 15 where the sampler includes a swab.

19. The method of claim 14 where a before sample from which the before value is measured is generated with an aggregating sampler.

20. The method of claim 13 where the enumerations are by qPCR.
